(19) United States
(12) Patent Application Publication
(10) Pub. No.: US 2005/0142097 A1
(43) Pub. Date: Jun. 30, 2005

(54) Multiple active drug resin conjugate

(76) Inventors: Deepak Thassu, Fairport, NY (US); Paul Hafey, Victor, NY (US)

Correspondence Address: PRICE HENEVELD COOPER DEWITT & LITTON, LLP, 695 KENMOOR, S.E., P O BOX 2567, GRAND RAPIDS, MI, 49501, US

(21) Appl. No.: 10/747,509

(22) Filed: Dec. 29, 2003

(51) Int. Cl. A61K 31/785

(52) U.S. Cl. 424/78.12

(57) ABSTRACT

A combination pharmaceutical preparation including two different active drugs of the same ionic charge conjugated with a single resin particle, without one significantly displacing the other, and without retarding the initial availability of either active. Also, methods for the manufacture of a multiple active drug resin conjugate, and for the in vivo release of a combination of pharmaceutically active drugs from a multiple active drug resin conjugate.

### FIELD OF THE INVENTION

This invention relates to drug resin conjugate compositions and a method of producing these compositions.

### BACKGROUND OF THE INVENTION

Pharmaceutical compositions including an active drug bound to an ion exchange resin have been known for many years. An ion exchange resin is an ionic, or charged, compound which has binding sites that can bind an ionic drug. Either a cationic or an anionic exchange resin can be used depending on whether the drug to be bound is acidic or basic. A basic drug is bound to a cationic exchange resin and an acidic drug is bound to an anionic exchange resin. Conjugation between the drug and the ion exchange resin particles result from ionic bonds between oppositely charged species because of their mutual electrostatic attraction. The conjugation of an active drug with an ion exchange resin forms a composition known as a “drug-resin complex.”

Ion exchange resins contain two principle parts: a structural portion consisting of a polymer backbone or matrix and a displaceable, functional portion, which is the ion-active group to which the drug is bound. The functional group may be acidic (sulfonic or carboxylic) or basic (usually an amine). Thus, drugs with the appropriate charge will bind to functional sites on the resin. The polymer chains are typically crosslinked with a crosslinking agent such as a divinyl or polivinyl compound. The crosslinking agent often is divinylbenzene. Particle size and the amount of crosslinking can vary for different resins.

Cationic ion exchange resins have negatively charged, or anionic, binding sites. The anionic binding sites are bonded to displaceable cationic groups. Cationic drugs are positively charged and tend to displace the cationic groups, typically becoming bonded to the resin by ionic bonds. The active drug is bound within the matrix of the resin. Since basic drugs are basically cationic, cationic exchange resins are often used to prepare drug-resin complexes with basic drugs. Typical approaches to forming a drug resin complex are to react the sodium salt of a cationic ion exchange resin with a cationic drug or to react the base form of the drug with the acid form of the cationic ion exchange resin.

Anionic exchange resins have positively charged, or cationic, binding sites. Anionic drugs are negatively charged and tend to displace the anionic groups located on the resin. Since acidic drugs are generally anionic, anionic exchange resins are frequently used to prepare drug resin complexes for acidic drugs.

Complexing an active drug with an ion exchange resin is referred to as “loading.” Loading of the drug on the resin can be accomplished by a number of techniques. For example, the active drug can loaded in a batch where the active drug is mixed with the resin for sufficient time to obtain the necessary amount of loading. Alternatively, a solution of the drug can be passed through a column of resin until the required loading has been completed.

In the gastrointestinal tract, a reverse ion exchange reaction takes place to release the drug. Namely, the many ions present in the digestive tract of the patient bind to and exchange for and displace the active drug from the resin and release the drug. For coated conjugates, release of the drug active from the drug-resin complex is a three-step process: (1) diffusion of gastrointestinal tract ions through the coating and into the polymer matrix, (2) release of the active drug from the resin complex, and (3) diffusion of the drug through the coating and into the gastrointestinal tract.

Drug resin complexes have advantages over drugs in pure form for several reasons. Complexing an active drug with a resin often improves the taste or smell as compared to the active drug alone. Further, active drugs often are conjugated to resins to enhance delivery of the active drug. Specifically, complexing an active drug with a resin can affect the rate at which the drug dissolves in the digestive system of a patient. While an active drug may dissolve at a fast rate which irritates or is harmful to the patient, a drug resin from a drug complex often will dissolve out of the complex more slowly than the active drug alone will dissolve. Rapid dissolution of an active drug can be problematic for a patient, especially when the active drug is most effective when delivered over a prolonged period of time. A controlled release drug preparation delivers drugs in a manner that effectively maintains plasma levels of the active drug over a period of time that is longer than that given by the drug in its pure form. Uncoated drug resin complexes, however, provide a relatively short delay of drug release which is limited by variation in resin particle size and crosslinkage of the resin.

Sustained or prolonged release of an active drug from a drug resin complex can be further controlled by coating the drug resin complex. After administration, the drug is slowly released from the resin over time thereby providing constant or near constant delivery of the drug to a patient. Further, it is known to coat drug resin complexes with an enteric coating so that the ion exchange occurs in the gastrointestinal tract rather than in the stomach.

Formulations including more than one active drug conjugated to more than one resin also are known. In such formulations, each active drug is ionic and of the same ionic charge. A pharmaceutical preparation including more than one active drug is referred to as a “combination” drug product. In U.S. Pat. No. 4,762,709, Sheumaker taught the undesirability of allowing two ionic active drugs to conjugate to the same resin particle. Sheumaker “postulates” that a second active drug of like charge to a first active drug binds to the same ion exchange resin and “exchanges upon the resin particle with the drug previously bound thereby causing an increase in the amount of unbound drug in the formulation as well as producing a change in the dissolution profile (e.g., the amount of drug in solution versus time) of the previously bound drug.” (Col. 2, lines 3-9). In a formulation of chlorpheniramine with a coated pseudoephedrine-resin complex, Sheumaker describes a “substantial decrease in the availability of pseudoephedrine [the first resin-bound active drug] during the first hour and a half after dosage and a substantial increase in availability thereafter as compared with the results obtained with pseudoephedrine alone.” (Col. 3, lines 31-35). As a solution, Sheumaker discloses binding each active drug to “its own” resin (Col. 2, lines 9-10), to prevent the two actives from binding to the same resin particles.

### SUMMARY OF THE INVENTION

In the present invention, it has been discovered that a combination pharmaceutical preparation can be prepared in which two different active drugs of the same ionic charge are conjugated with a single resin particle, without one significantly displacing the other, and without retarding the initial availability of either active. Also, the invention is directed to methods for the manufacture of the multiple active drug resin conjugate, and for the in vivo release of a combination of pharmaceutically active drugs from the multiple active drug resin conjugate. These and other objects, aspects, and features of the invention will be more fully understood and appreciated by reference to the written specification.

### BRIEF DESCRIPTION OF THE DRAWINGS

FIG. 1 is a graph of a Mean Chlorpheniramine Plasma Concentration Versus Time Course Following Single Dose of 10 mL Extended Release Suspension and 5 mL Immediate Release Solution Administered Every 6 Hours for 2 Consecutive Doses, Fasting Conditions; and

FIG. 2 is a graph of Mean Codeine Plasma Concentration Versus Time Course Following Single Dose of 10 mL Extended Release Suspension and 5 mL Immediate Release Solution Administered Every 6 Hours for 2 Consecutive Doses, Fasting Conditions.

### DETAILED DESCRIPTION OF THE PREFERRED EMBODIMENT

In the preferred embodiment, two ionic drugs of like charge are conjugated to the same resin particles. They can be slurried and conjugated with the resin particles simultaneously, or either can be slurried and conjugated first, followed by conjugation of the other.

In one preferred embodiment, one active is conjugated with the resin, and the particles are then coated with an extended release coating. The resulting coated particles are then slurried with the second active, with the input of energy. The resulting “two active” particles are then formulated into a suspension, along with any optional ingredients. Energy is then applied to the final suspension product.

Resins

A wide range of cationic (for the basic drugs) or anionic (for the acidic drugs) exchange resins can be used to form a multiple active drug resin complex. Ion exchange resins suitable for use in the invention comprise a pharmacologically inert organic or inorganic matrix containing covalently bound functional groups that are ionic or capable of being ionized under appropriate conditions of pH. The functional portion of the resin may be strongly acidic (e.g., sulfonic acid, phosphoric acid), weakly acidic (e.g., carboxylic acid), strongly basic (e.g., primary amine), or weakly basic (e.g., quateranary ammonium).

In a preferred embodiment of the invention, a cationic sulfonate polistirex ion exchange resin is conjugated with more than one basic active drug. Such a preferred resin is Amberlite IRP-70.

Both regularly and irregularly shaped particles may be used as resins. Regularly shaped particles are those particles that substantially conform to geometric shapes, such as spherical, elliptical, cylindrical, and the like. Irregularly shaped particles are particles that are not regular in shape, such as particles with amorphous shapes or particles that include channels or other distortions.

The resin particle size used varies at least in part as a function of the delivery system to be used. Generally, the size of the ion-exchange particles is from about 30 microns to about 500 microns. For suspensions, prior artisans have typically used resin particles which are 40 to 150μ. For tablets, larger particles (e.g., up to about 1000 microns) can be utilized. However, the particles eventually become so large that they are inefficient, i.e., the weight ratio of active to resin becomes too small. That is because the available bonding charge per unit of weight of resin particle becomes smaller as particles become larger, and more of the charge is buried in the interior of the particle. In other words, the exchange capacity of the resin is diminished.

Active Drugs

Conjugated to the resin particles are pharmaceutically active drugs that are suitable for the treatment of various disorders, for example, antitussive expectorants, decongestants, bronchodilators, antihistamines, digestive tract antispasmodics, drugs for the treatment of central nervous system disorders, anti-anxiety drugs, antidepressants, coronary dilators, anti-arrhythmics, calcium antagonists, hypotensive drugs, peripheral vaso dilators or vaso constrictors, antibiotics, chemotherapeutic drugs, anti-tuberculosis drugs, anti-protozoan drugs, alpha-adrenergic agonists and blockers, beta adrenergic agonists and blockers, narcotic and non-narcotic analgesics, anorexics, anti-anginals, anti-asthmatics, anti-cholinergics, anti-fungals, anti-inflammatories, anti-spasmodics, anti-ulceratives, anti-virals, anxiolytics, calcium channel blockers, dopamine receptor agonists and antagonists, narcotic antagonists, protease inhibitors, respiratory stimulants, retroviral protease inhibitors, reverse transcriptase inhibitors, sedatives, and anti-hypertensives. Examples of particular drugs are codeine, chlorpheniramine, pseudoephedrine, phenylpropanolamine, dextromethorphan, and propanolol.

Drugs which exist in ionic form in a semi-polar or polar solvent, such as water, are potential candidates for use in this invention. Drugs useful in the present invention may be either acidic, basic, or amphoteric. Examples of acidic drugs that can be used in the present invention include, but are not limited to, dehydrocholic acid, diflunisal, ethacrynic acid, fenoprofen, furosemide, gemfibrozil, ibuprofen, naproxen, phenytoin, probenecid, sulnadic, theophyllline, salicylic acid, acetylsalicylic acid. Examples of basic drugs that can be used in the present invention include, but are not limited to acetophenazine, amitriptyline, amphetamine, benztropine, biperiden, bromodiphenhydramine, brompheniramine, carbinoxamine, chloperastine, chlorcyclizine, chlorpheniramine, chlorphenoxamine, chlorpromazine, clemastine, clomiphene, clonidine, codeine, cyclizine, cyclobenzaprine, cyproheptadine, desipramine, dexbrompheniramine, dexchlorpheniramine, dextroamphetamine, dextromethorphan, dicyclomine, diphemanil, diphenhydramine, doxepin, doxylamine, ergotamine, fluphenazine, haloperidol, hydrocodone, hydroxychloroquine, hydroxyzine, hyoscyamine, imipramine, levopropoxyphene, maprotiline, meclizine, mepenzolate, meperidine, mephentermine, mesoridazine, methadone, methylephedrine, methdilazine, methscopolamine, methysergide, metoprolol, nortriptylene, noscapine, nylindrin, orphenadrine, papaverine, pentazocine, phendimatrazine, phentermine, phenylpropanolamine, pyrilamine, tripelennamine, triprolidine, promazine, propoxyphene, propanolol, pseudoephedrine, pyrilamine, quinidine, scopolamine, dextramethorphan, chlorpheniramine, and codeine. Amphoteric drugs that can be used in the present invention include, for example, aminocaproic acid, aminosalicylic acid, hydromorphone, isoxsurpine, levorphanol, melphalan, morphine, nalidixic acid, and paraaminosalicylic acid.

The pharmaceutical composition of the present invention is administered at dosage levels of the active drug or drugs that will vary according to factors such as age, weight, and condition of the patient. Formulations comprising different active drugs will be administered according to usual daily dosages of the individual drugs.

Active Drug-Resin Complex

Adsorption or conjugation of an active drug onto an ion exchange resin particle to form an active drug resin complex is well known, as shown in U.S. Pat. No. 2,990,332 (Keating) and U.S. Pat. No. 4,221,778 (Raghunathan). Such a drug resin conjugate is prepared by slurring the exchange resin particles in a solution containing the active drug. The ideal weight ratio of active drug to resin particles will vary as a function of the charge to weight ratio of the active, and the exchange capacity of the resin particles (e.g., resin particle size, crosslinkage). Other factors in determining the ratio of drug to resin are the reaction conditions and the final dosage form. To a degree, a greater weight of a higher molecular weight active will bind to a given resin than will a lower molecular weight active, using the same charge per molecule for each active.

The amount of drug that can be loaded onto the resin will typically range from about 1 percent to about 90 percent by weight of the drug resin particles, although 15 to 50 percent by weight is the normal range.

The present invention includes two or more active drugs bound to a single resin particle. Any of the active drugs identified above can be utilized as the second (or subsequent) active drug to be added to the drug resin complex. However, the second (or subsequent) active drug must be of the same charge as the first active drug loaded on the resin. The molar ratio of actives to one another will vary as a function of target formulation and physical and chemical characteristics of the drugs used, but in general can vary from about 1:1 to about 10:1.

In a preferred embodiment of the invention, codeine and chlorpheniramine are the active drugs, both of which are basic and are conjugated to a single sulfonate polistirex ion exchange resin. Preferably, codeine and chlorpheniramine are provided in a molar ratio of from about 5:1 to about 8:1. In one preferred embodiment, the codeine and chloropheneramine product comprises codeine base at 40 mg and Chlorpheneramine base at 5.6 mg. A codeine to chlorpheniramine molar ratio of from about 1:1 to about 10:1 also can be used. Because these two actives are fairly close in weight, the weight ratios used will be about the same as the molar ratios. A preferred weight ratio of the sulfonate polistirex resin to codeine is about 5:1; and a preferred weight ratio of the sulfonate polistirex resin to chlorpheniramine is about 42:1. A preferred weight ratio of the multiple active resin complex (resin/codeine/chlorpheniramine) to codeine is about 6:1; and a preferred weight ratio of the multiple active resin complex (resin/codeine/chlorpheniramine) to chlorpheniramine is about 51:1.

After a first active drug (e.g., codeine) is loaded onto an ion exchange resin, some exchange sites on the resin still include a sodium or hydrogen ion. When added, the second active drug (e.g., chlorpheniramine) is attracted to the exchange sites bearing a sodium or hydrogen ion, and the second active drug exchanges with the sodium or hydrogen ion. However, with some vehicles, minimal amount of one active may disassociate from the resin and move into the suspension. In a preferred embodiment of the invention, with codeine as the first active drug and chloropheneramine as the second active drug, 2-3 percent of the codeine comes off of the codeine-resin complex and moves into suspension. This small amount of disassociation is believed to be caused by the inherent properties of various surface active agents (e.g., polyethylene glycol), i.e., the disassociation occurs due to a “vehicle effect.

In another embodiment of the invention, two or more acidic drugs are loaded onto an anionic ion exchange resin, without either acidic drug exchanging on the resin for another acidic drug. For example, omeprazole is first conjugated with an anionic exchange resin. Such anionic exchange resin particles are commercially available as Duolite® resin and resin from Purolite International Limited, both of which are cholestyramine, a synthetic anionic exchange polymer in which quaternary ammonium groups are attached to a polystyrene-divinylbenzene copolymer. Another acidic drug, selected from the group including esomeprazole, lansoprazole, pantoprazole, rabeprazole or leminoprazole, is then slurried with the conjugate to form a final product.

Impregnation

In order to permit particles to retain their geometry and allow the effective application of diffusion barrier coatings to such particles, active drug-resin complexes are impregnated with a solvating agent. Polyethylene glycol, a hydrophilic agent, is a preferred solvating agent. Other impregnating agents include mannitol, lactose, methylcellulose, hydroxypropylmethylcellulose, sorbitol, polyvinylpyrlodone, carboxypolymethylene, xantham gum, propylene glycol alginate, and various combinations of these solvating agents.

Diffusion Barrier Coating

Controlled release of an active drug from a drug resin complex can be achieved through the application of a diffusion barrier coating to a drug resin complex, provided that the concentration of active drug is above a critical value. Coating materials may be natural or synthetic film formers, along with plasticizers, pigments, and other substances which alter the characteristics of the coating. In general, the major components of the coating should be insoluble in and permeable to water. The coating also should be ion permeable. Preferably, the water permeable diffusion barrier is a cellulose ether, more preferably selected from the group consisting of ethylcellulose, methylcellulose, hydroxypropylmethylcellulose, other cellulose polymers, and mixtures thereof. More preferably, the diffusion barrier is ethylcellulose. Preferred synthetic barriers are methacrylic polymers and copolymers.

The inclusion of an effective amount of plasticizer in the aqueous dispersion of the polymer will further improve the physical characteristics of the film coating. Generally, the amount of plasticizer included in the coating solution depends on the concentration of the film former, and the plasticizer preferably is from about 1 to about 50 percent by weight of the film former. Such a plasticizer can be, e.g., vegetable oil, dibutylsebacate, diethylsebacate, diethylphthalate, tricetin, or propyleneglycol, with vegetable oil the preferred plasticizer.

Depending upon the desired release profile of the active drugs, the coating weight and coating thickness may be varied. The coating materials can be applied as a suspension in an aqueous fluid or as a solution in organic solvents. Any coating procedure which provides a contiguous coating on the complexed particle may be used. One example is a fluid bed coating apparatus having a Wurster configuration.

Enteric Coating

In order to prevent the active ingredients in the composition from disassociating with the complex or from disintegrating in the stomach, it is known in the art to apply an enteric coating either to the barrier-coated initial drug resin complex or directly on the initial drug resin complex. The enteric coating allows the active ingredients to be released once the dosage form is in the small intestinal tract. Materials useful for enteric coating should be insoluble in a low pH medium typically having a pH less than 3.5, but soluble in a higher pH medium, typically greater than 5.5.

Commonly used enteric coatings include cellulosic materials such as cellulose acetate phthalate, cellulose acetate trimellitate, cellulose acetate succinate, hydoxypropylmethylcellulose phthalate, hydroxypropylmethylcellulose acetate succinate, and carboxymethylethylcellulose. Other non-cellulosic polymers may be used, e.g., copolymers of methacrylic acid and methylmethacrylate or ethylacrylate, terpolymers of methacrylic acid, methacrylate, and ethylacrylate, and polyvinyl acetate phthalate. The amounts and types of polymers in the enteric coating and the thickness of the enteric coating applied to the drug resin particles may be selected to control the rate of drug released.

Liquid Carrier

The multiple active resin complex is formulated into a suitable liquid vehicle containing water and, as desired, additional solvent thickners, preservatives, coloring agents, flavoring agents, solubilizers, dispersants, and other typical adjuvants, all of which must be pharmaceutically acceptable and are known in the art. Typical carriers include simple aqueous solutions, syrups, dispersions, and suspensions, and aqueous-based emulsions such as the oil-in-water type. The preferred carrier of the present invention is suspension of the pharmaceutical composition in an aqueous vehicle containing sufficient suspending agent. Suitable suspending agents include Avicel RC-591, NF (a microcrystalline cellulose/sodium carboxylmethylcellulose mixture available from FMC), guar gum, and the like. The amount of water can vary and depends upon the total weight and volume of the drug resin complex and other non-active ingredients.

Typical liquid formulations also may contain a co-solvent, e.g., propylene glycol, glycerin, sorbitol solution, and the like to assist solubilization and incorporation of water insoluble ingredients, such as flavorings, into the composition.

The suspension of the present invention is contained in a bottle or other multi-use vehicle that is repeatedly opened and closed. During such use, microbes can gain access to and contaminate the suspension. Accordingly, it is well known to use various microbicidal agents to kill such microbes. Preferably, these agents include methylparaben and propylparaben. Additionally, various chelating agents are employed to prevent microbial growth. One preferred bacteriostatic agent is edetate disodium. Microbicidal and bacteriostatic compounds are surface active agents that effect permeability of the diffusion barrier coating.

Method of Manufacture

Multiple active drug resin conjugates can be manufactured using equipment well known in the art. Methods of manufacture can be varied, depending upon the type and amount of resin, active drugs, and coating. As discussed above, and as demonstrated in Example 1 below, the two actives can be slurried and conjugated with the resin particles simultaneously or sequentially in either order.

In one preferred embodiment used in Example 2 and 3 below, a first active drug, codeine phosphate, is conjugated with a sulfonate polistirex ion exchange resin (e.g., Amberlite IRP-70, washed sodium cycle), treated with polyethylene glycol, and the treated conjugate is coated with a diffusion barrier coating of ethylcellulose and vegetable oil. More specifically, codeine phosphate is dissolved in water; Amberlite IRP-70 is added; a solution of sodium hydroxide, disodium edetate, and polyethylene glycol are mixed with the codeine-polistirex complex resulting in a polyethylene-treated codeine-polistirex conjugate; the polyethylene-treated codeine-polistirex conjugate is dried to a final moisture of 5-7%, preferably 6%; the dried polyethylene-treated codeine-polistirex conjugate is then screened, preferably with a 20-mesh screen. Using a fluid bed coating apparatus having a Wurster configuration, the polyethylene glycol-treated codeine-polistirex conjugate then is coated at a level of about 12% with a solution of ethylcellulose and vegetable oil. The coated conjugate is then screened, preferably with a 40-mesh screen.

Further, in this preferred embodiment, the coated codeine-polistirex conjugate is slurried with a second active drug, chlorpheniramine maleate, with a first application of energy. Namely, Polysorbate 80, citric acid, edetate disodium, and the chlorpheniramine maleate are mixed in solution; subsequently, the coated codeine polistirex product is added into this solution and mixed for 18-30 hours (preferably 24 hours) while maintaining the temperature at about 55-65 degrees centigrade, preferably 60° centigrade. This first application of energy modulates permeability of the diffusion barrier coating allowing the second active drug to migrate through the diffusion barrier coating to the matrix of the codeine-polistirex conjugate. Further, the energy input provided by this heating step yields an even, predictable dissolution or release profile for each active drug as well as a consistent release profile for each drug with aging. In addition to the application of energy by heating, energy can be imparted to the system by stirring vigorously for an extended period of time thereby accelerating such migration of the second active drug through the diffusion barrier coating. Further, a combination of heat (high energy) and stirring (generally a lower form of energy) also can be employed together to accelerate migration of the second active drug through the diffusion barrier coating.

Finally, additional ingredients, if any, are blended in, and the codeine-chlorpheniramine-polistirex product is subjected to a second application of energy in an amount and for a time sufficient to yield a product with a stable drug release profile. In a preferred embodiment of the invention, in a separate batch vessel, sucrose NF is mixed until dissolved in water; dipropylene glycol, methyl paraben, propylparaben, and xantham gum are added to the batch vessel and mixed until uniform; glycerin is then added to the batch vessel which is heated to 55-65 degrees centigrade, preferably 60° centigrade; the codeine-chlorpheniramine-polistirex conjugate is added to the batch vessel and heat continued to be applied to the suspension at about 55-65 degrees centigrade, preferably 60° centigrade for about 4-10 hours, preferably, 6 hours. This second application of energy establishes a final stable state for the diffusion barrier coating keeping the chloropheneramine and codeine within the diffusion barrier coating, and also evenly distributing the microbicidal and bacteriostatic agents to reduce their effect on the diffusion barrier coating. This results in a stable product with 1-2% change over a 1-34 month period at a 25 degrees Centigrade aging condition.

The present invention is further illustrated by the following examples, which are not intended to be limiting. It is to be understood by those skilled in the art that modifications and changes can be made thereto without departing from the spirit and scope of the invention.

### EXAMPLES

### Example 1

### Dissolution Study of Codeine/Chloropheneramine Formulation

The release profiles for codeine/chlorpheniramine resin conjugates produced by three separate processes were studied, demonstrating that one active drug does not exchange for the other active drug. Specifically, experiments were conducted as follows: (a) codeine and chlorpheniramine were simultaneously conjugated to a single resin particle (Lot #001); (b) codeine was conjugated first to a single resin particle and then chlorpheniramine was conjugated to the codeine-resin particle (Lot #004); and (c) chlorpheniramine was first conjugated to the resin particle, with codeine subsequently conjugated to the chlorpheniramine-resin particle (Lot #007).

Sheumaker U.S. Pat. No. 4,762,709 would suggest, using the method to produce Lot #004, that some of the chlorpheniramine would displace the codeine on the resin. That is not the case with the present invention. Specifically, 16.37 percent codeine (as a percent of total codeine-chlorpheniramine-resin weight) is used to prepare Lot #004, along with 1.94 percent chlorpheniramine. Analytical assay results of the resultant multiple active resin product of Lot #004 demonstrate 16.6 percent codeine and 2.2 percent chlorpheniramine confirming that all of the codeine used to produce the product was incorporated into the resin conjugate, i.e., the quantity of codeine added in to manufacture the product substantially equals the quantity of codeine “out” in the final product. There was no competition by chlorpheniramine for the sites occupied by codeine.

The results with respect to Lot #004 were confirmed by Lots #001 and #007. Specifically, with respect to Lot #001, the drug ratio added was 16.37 percent codeine and 1.94 percent chlorpheniramine, while the analytical assay results showed 16.00 percent codeine and 2.0 percent chlorpheniramine. With respect to Lot #007, the drug ratio for codeine was 16.37 percent and for chlorpheniramine 1.94 percent, while the analytical assay results showed 16.6 percent codeine and 2.10 percent chlorpheniramine.

### Example 2

### Biological Availability Study of Codeine/Chloropheneramine Formulation

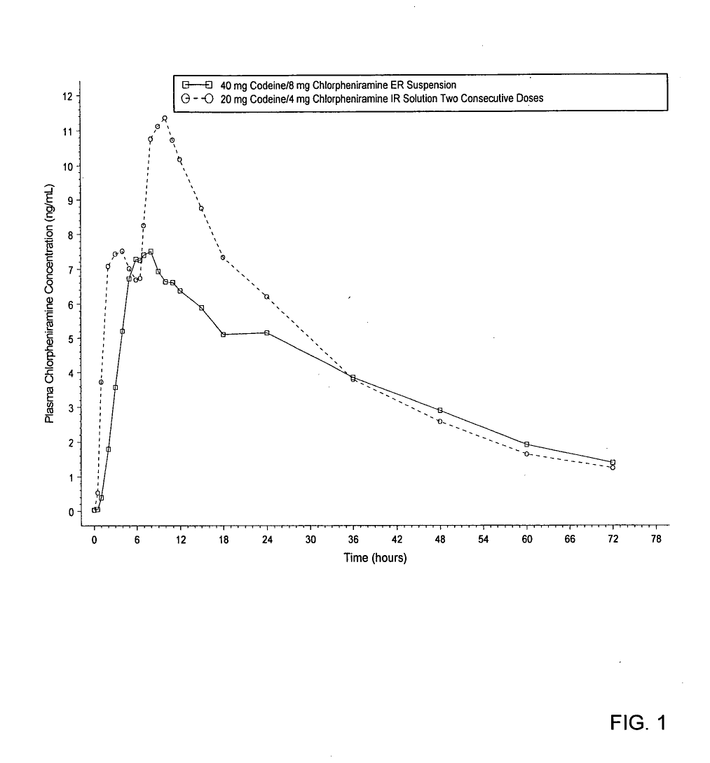

An in vivo study was conducted using a codeine/chloropheneramine resin conjugate prepared as discussed above. FIG. 1 shows the mean chlorpheniramine plasma concentration versus time of a single dose of 10 milliliters of extended release suspension (prepared according to the method described above), including 40 milligrams of codeine and 8 milligrams of chlorpheniramine. FIG. 1 also shows the mean chlorpheniramine plasma concentration versus time of two consecutive doses (6 hours apart) of 5 milliliters of immediate release solution, with each dose including 20 milligrams of codeine and 4 milligrams of chlorpheniramine. The immediate release product was prepared by dissolving chlorpheniramine salt with polyethylene glycol and sweetener in water. The same amount of chlorpheniramine is loaded for both the extended release and immediate release products. No resin of any kind is added in the immediate release formulation; and in the extended release product, the only chlorpheniramine present is bound to the resin.

The chlorpheniramine plasma concentration over time shows similar bioavailability for the extended release as compared to the double-dosed immediate release product, confirming that all of the chlorpheniramine used to produce the extended release product was incorporated into the resin conjugate. That is, the quantity of chlorpheniramine added in to produce the product must have been conjugated with the resin because it all comes out in the patient.

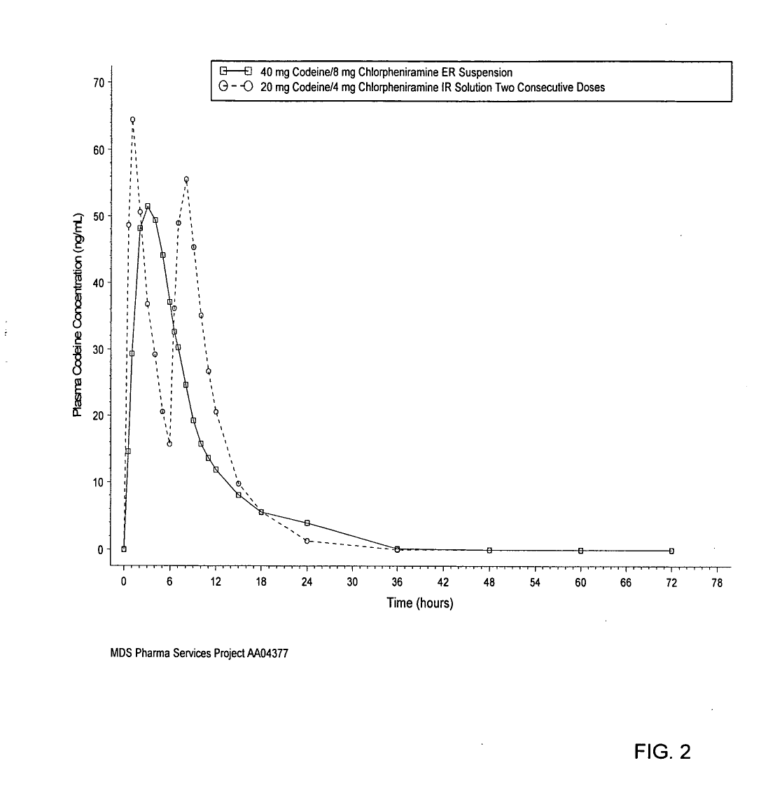

The mean codeine plasma concentrations versus time for this study are shown in FIG. 2. As with chlorpheniramine, the codeine plasma concentration over time shows similar bioavailability for the extended release as compared to the double-dosed immediate release product, confirming that all of the codeine used to produce the extended release product was incorporated into the resin conjugate. Namely, the quantity of codeine added in to produce the product must have been conjugated with the resin because it all comes out in the patient.

### Example 3

Using the same codeine/chloropheneramine formulations described above, in-vitro drug release data demonstrates even, predictable dissolution profiles of the first active (codeine) and second active (chlorpheniramine), which release profiles are stable over time. Tables I and II show these results.

TABLE I
Codeine Release

| | | 3 M, | 6 M, | 9 M, | 1.5 M, | 3 M, | 6 M, |
|---|---|---|---|---|---|---|---|
| | Initial | 25 C.* | 25 C. | 25 C. | 40 C. | 40 C. | 40 C. |
| 1-Hour | 54 | 54 | 54 | 54 | 55 | 56 | 54 |
| 3-Hour | 75 | 75 | 75 | 75 | 77 | 77 | 77 |
| 6-Hour | 86 | 86 | 85 | 86 | 88 | 88 | 87 |
| 12-Hour | 93 | 93 | 93 | 94 | 95 | 95 | 94 |

*3 months aging at 25° C.

TABLE II
Chlorpheniramine Release

| | | 3 M, | 6 M, | 9 M, | 1.5 M, | 3 M, | 6 M, |
|---|---|---|---|---|---|---|---|
| | Initial | 25 C. | 25 C. | 25 C. | 40 C. | 40 C. | 40 C. |
| 1-Hour | 42 | 43 | 41 | 42 | 41 | 43 | 39 |
| 3-Hour | 65 | 64 | 63 | 63 | 63 | 65 | 62 |
| 6-Hour | 78 | 77 | 76 | 77 | 77 | 78 | 76 |
| 12-Hour | 88 | 88 | 87 | 88 | 87 | 90 | 87 |

The dissolution testing shown in Tables I and II was conducted using a release medium of hydrochloric acid, sodium chloride, and sodium dodecylsulfate. With respect to Table I, the drug release data for codeine shows even, predictable results, for example, at the initial testing, 54 percent dissolution in 1 hour, 75 percent dissolution at 3 hours, 86 percent dissolution at 6 hours, and 93 percent dissolution at 12 hours. The dissolution profile for chlorpheniramine, shown in Table II, also is even and predictable, for example, at the initial testing, 42 percent dissolution of chlorpheniramine at 1 hour, 65 percent at 3 hours, 78 percent at 6 hours, and 88 percent at 12 hours.

Further, Tables I and II show even, predictable drug release data at one and one half months, 3 months, 6 months, and 9 months, with only very minor variations. These results are shown at both 25° centigrade and at 40° centigrade. Thus, with aging of the product, the release profile is consistent, allowing use of the present invention in compliance with various stability requirements for pharmaceutical products.

In the foregoing description, it will be readily appreciated by those skilled in the art that modifications may be made to the invention without departing from the concepts disclosed herein. Such modifications are to be considered as included in the following claims, unless these claims by their language expressly state otherwise.

1. A drug resin conjugate comprising at least two different drug moieties conjugated onto a single resin particle, without any drug significantly displacing another drug on the resin particle.

2. The conjugate of claim 1 in which the molar ratio of the drug moieties to one another varies from about 1:1 to about 10:1.

3. The conjugate of claim 1 wherein the initial availability of either drug moiety is not retarded.

4. The conjugate of claim 1 wherein the resin particle is a cationic ionic resin exchange particle.

5. The conjugate of claim 4, wherein the resin particle is a sulfonate polistirex resin.

6. The conjugate of claim 1 wherein the conjugate is coated with a dissolution barrier.

7. The conjugate of claim 6 wherein the dissolution barrier coating is a cellulose ether.

8. The conjugate of claim 7 wherein the cellulose ether is ethycellulose, methylcellulose, or hydroxypropylmethylcellulose.

9. The conjugate of claim 6 wherein the dissolution barrier coating is a synthetic material.

10. The conjugate of claim 9 wherein the synthetic material is a methacrylic polymer or copolymer.

11. The conjugate of claim 6 wherein the coating includes a plasticizer.

12. The conjugate of claim 1 wherein the resin particle is a anionic ionic resin exchange particle.

13. The conjugate of claim 12, wherein the resin particle is a polystyrene-divinyl copolymer with a quaternary ammonium group.

14. The conjugate of claim 1 wherein the two drug moieties are similarly charged.

15. The conjugate of claim 1 wherein the two drug moieties are both basic.

16. The conjugate of claim 15 wherein the two drug moieties are codeine and chlopheniramine.

17. The conjugate of claim 16 wherein codeine and chlorpheniramine are provided in a molar ratio of from about 5:1 to about 8:1.

18. The conjugate of claim 17 wherein the conjugate is coated with a dissolution barrier.

19. The conjugate of claim 18 wherein the dissolution barrier coating is a cellulose ether.

20. The conjugate of claim 19 wherein the cellulose ether is ethycellulose, methylcellulose, or hydroxypropylmethylcellulose.

21. The conjugate of claim 18 wherein the dissolution barrier coating is a synthetic material.

22. The conjugate of claim 21 wherein the synthetic material is a methacrylic polymer or copolymer.

23. The conjugate of claim 18 wherein the coating includes a plasticizer.

24. The conjugate of claim 1 wherein the two drug moieties are both acidic.

25. The conjugate of claim 24 wherein the two drug moieties are selected from the group consisting of omeprazole, esomeprazole, lansoprazole, pantoprazole, rabeprazole or leminoprazole.

26. The conjugate of claim 1 wherein the conjugate includes an enteric coating.

27. The conjugate of claim 1 wherein the conjugate is coated with a dissolution barrier and with an enteric coating.

28. A method of making a multiple active drug resin conjugate comprising: simultaneously slurrying a first active drug and a second active drug with ion exchange resin particles.

29. A method of making a multiple active drug resin conjugate comprising: slurrying a first active drug with ion exchange resin particles, followed by slurrying the resulting conjugate with a second active drug.

30. A method of making a multiple active drug resin conjugate comprising: slurrying a coated first active drug-resin conjugate with a second active drug, with a first application of energy.

31. The method of claim 30 wherein the second active drug complexes with the first active drug-resin conjugate without significantly displacing the first active drug on the resin.

32. The method of claim 30 wherein the first energy applied is a first heating.

33. The method of claim 32 wherein the first heating is applied at a temperature from 55 to 65° centigrade for 18 to 30 hours.

34. The method of claim 30, further comprising the step of preparing a suspension of said multiple active drug resin conjugate, and conducting a second application of energy.

35. The method of claim 34 wherein the second energy applied is second heating.

36. The method of claim 35 wherein the second heating is applied at a temperature from 55 to 60° centigrade for an additional 6 to 10 hours.

37. The method of claim 36 wherein the conjugate is stable with aging.

38. A method of forming a codeine and chlorpheniramine drug resin complex comprising: slurrying codeine with ion exchange resin particles, and then coating the resulting drug resin conjugate particles with a coating, followed by slurrying the resulting coated conjugate with chlorpheniramine with the application of heat.

39. The method of claim 38 in which said heating is conducted at a temperature of from about 55 to about 650 centigrade for about 18 to about 30 hours.

40. The method of claim 39 which further comprises preparing a suspension of said multiple active drug resin conjugate, and heating the resulting suspension at a temperature and for a time sufficient to create a suspension exhibiting a drug release profile which remains stable with aging.

41. The method of claim 40 in which said heating of said suspension is conducted at a temperature from about 55 to about 60° centigrade for from about 6 to about 10 hours.

42. The method of claim 38 which further comprises preparing a suspension of said multiple active drug resin conjugate, and heating the resulting suspension at a temperature and for a time sufficient to create a suspension exhibiting a drug release profile which remains stable with aging.

43. A method of treating patients comprising the step of providing a drug resin conjugate comprising two different drug moieties conjugated onto a single resin particle, without one drug significantly displacing the other drug on the resin particle.

44. The method of claim 43 wherein the initial availability to the patient of either drug moiety is not retarded.

45. The method of claim 44 wherein the resin particle is a cationic ionic resin exchange particle.

46. The method of claim 43 wherein the two drug moieties are codeine and chlorpheniramine.

47. The method of claim 41 wherein codeine and chlorpheniramine are provided in a molar ratio of from about 5:1 to about 8:1.

48. The method of claim 44 wherein the resin particle is a anionic ionic resin exchange particle.

49. The conjugate of claim 43 wherein the conjugate includes a diffusion barrier coating.

50. The conjugate of claim 49 wherein the conjugate also includes an enteric coating over said diffusion barrier coating.

51. The conjugate of claim 43 wherein the conjugate also includes an enteric coating.
